(19) United States
(12) Patent Application Publication
(10) Pub. No.: US 2013/0102879 A1
(43) Pub. Date: Apr. 25, 2013

(54) Method For Correcting Susceptibility-Induced Image Artifacts In MRI After Prospective Motion Correction

(71) Applicants: Julian Maclaren, Freiburg (DE); Rainer Boegle, Munich (DE); Maxim Zaitsev, Freiburg (DE)

(72) Inventors: Julian Maclaren, Freiburg (DE); Rainer Boegle, Munich (DE); Maxim Zaitsev, Freiburg (DE)

(73) Assignee: UNIVERSITAETSKLINIKUM FREIBURG, Freiburg (DE)

(21) Appl. No.: 13/639,869

(22) PCT Filed: Apr. 14, 2010
(86) PCT No.: PCT/EP2010/002278

§ 371 Date: Jan. 7, 2013

(51) Int. Cl. A61B 5/055; A61B 5/00

(52) U.S. Cl. CPC A61B 5/055; A61B 5/0035
USPC 600/411; 600/410

(57) ABSTRACT

A method of magnetic resonance imaging (MRI) is characterized by the following steps: a) forming a susceptibility model (305, 403) of at least a part of a subject (S), including an imaged body part (203), by using a structural magnetic resonance image (301) of the part of the subject (S) and/or prior knowledge of the anatomy of the subject (S); b) computing susceptibility-induced field deviations (404) present in the imaging volume at each time MR signals are acquired using the susceptibility model (305, 403) and the knowledge of a monitored position and monitored orientation (401) of the part of the subject (S) at that time; c) using the information about the susceptibility-induced field deviations (404) derived in b) for image correction (406), in particular correction of image distortions and/or intensity modulations. The quality of magnetic resonance imaging of moving subjects is thereby improved.

The invention relates to a method of magnetic resonance imaging (MRI), wherein a body part of an animal or human subject in an imaging volume is imaged by acquiring a plurality of spatially-encoded MR signals from the imaging volume where susceptibility-induced field deviations reduce the homogeneity of the main magnetic field B₀,

and wherein a prospective motion correction is applied, updating the imaging volume between the acquisition of the said spatially-encoded MR signals based on a monitored position of the body part.

Such a method is known from S. Thesen, O. Heid, E. Mueller, and L. R. Schad, “Prospective acquisition correction for head motion with image-based tracking for real-time fMRI,” Magn Reson Med, vol. 44, pp. 457-463, September 2000 (Ref. [1]) and M. Zaitsev, C. Dold, G. Sakas, J. Hennig, and O. Speck, “Magnetic resonance imaging of freely moving objects: prospective real-time motion correction using an external optical motion tracking system,” Neuroimage, vol. 31, pp. 1038-1050, Jul 2006 (Ref. [2]).

MRI (magnetic resonance imaging) is a well-known imaging method, which manipulates magnetic spins in the object to generate an image. This is achieved using a strong static field (known as the B₀ field) for spin polarisation, gradient fields to provide spatial localisation and a radio frequency (RF) field to generate signal. MRI has been extensively applied to the field of medicine, both for clinical routine imaging, and for the study of the human body, including the brain.

Subject motion (note that the subject is typically a human or an animal) is a major problem in MRI. In clinical routine imaging it can render images non-diagnostic, resulting in wasted scan time. In functional MRI (fMRI) studies of the human brain, motion can produce false activations and misleading results. Functional MRI uses the echo planar imaging (EPI) technique to rapidly acquire a series of images for later analysis. In the case of subject head motion, the images in this series are no longer aligned. To some extent, they can be realigned in post-processing; however, this fails to correct for spin history effects or movement of the subject outside of the imaging volume. Thus, studies that require, or result in, subject head movement cannot be performed. This limits the study of certain aspects of human cognition and behaviour.

Prospective motion correction is a promising new motion-correction technique. The technique works by monitoring the subject position and simultaneously updating the imaging volume. Subject position can be monitored using image registration of the latest acquired images [1] or by using an external tracking system [2]. Although prospective motion correction has been shown to be effective, it does not account for the time-varying image distortions that are caused by moving susceptibility interfaces distorting the main magnetic field (B₀). If motion amplitude is small, B₀ homogeneity is only slightly affected. However, for larger motion amplitudes, B₀ changes are considerable. As a result, applying prospective motion correction is not a complete solution to the problem of subject motion.

### Object of the Invention

It is the object of the invention to further improve the quality of magnetic resonance imaging of moving subjects.

### Short Description of the Invention

This object is achieved, in accordance with the invention, by a method as introduced in the beginning, characterized by the following steps:

a) forming a susceptibility model of at least part of the subject, including the imaged body part, by using a structural magnetic resonance image of the said part of the subject and/or prior knowledge of the anatomy of the subject;
b) computing the susceptibility-induced field deviations present in the imaging volume at each time MR signals are acquired using the susceptibility model and the knowledge of a monitored position and monitored orientation of the said part of the subject at the said time;
c) using the information about the susceptibility-induced field deviations derived in b) for image correction, in particular correction of image distortions and/or intensity modulations.

The present invention provides a method for the operation of a magnetic resonance imaging device which allows the correction of both motion and susceptibility-induced image artefacts. This is, in particular, an important step towards allowing fMRI during significant head motion.

In accordance with the invention, a susceptibility model of at least part of the subject including the imaged body part (the imaged body part is also known here as the “volume of interest”) within and near to the imaging volume (which encompasses the volume of interest in the subject) is used to predict, and allow correction of, artefacts caused by susceptibility-induced field inhomogeneities. These artefacts are dependent on the position and orientation of the said part of the subject including the imaged body part, which is obtained using an external tracking system, navigator echoes, or a related method.

The method relies on the accurate prediction of the B₀ field distribution for each possible position and orientation of the said part of the subject (further also referred to as “object”) during imaging. Measuring the field using field mapping techniques for each of these positions is impractical, due to the large number of possible positions and the time required to generate each field map. To avoid this, the field distribution is computed.

In a first step, the object is typically imaged using a structural sequence, and then segmented, either automatically or manually, into a range of material types (or tissue types, in most cases). In accordance with the invention, a simplified segmentation procedure may be used, which distinguishes only those tissue types relevant to generation of the final field. This is dependent on the relative magnetic susceptibilities of the different tissues. In some situations a highly simplified segmentation, where voxels are classified as either ‘air’, ‘bone’ and ‘water/other tissue’ already produces accurate field estimation. Prior knowledge of anatomy (from a digital brain atlas, for example) may be used to improve the segmentation. The susceptibility model formed in this step contains information about the spatial distribution of susceptibilities (and in particular location of susceptibility borders) in the object.

In a second step, the susceptibility distribution obtained from the first step is used to predict inhomogeneities in the B₀ field, e.g. by combining the susceptibility distribution with the field estimation method of Koch et al. [3]. Preferably, and in accordance with the invention, information from the prospective motion correction system is incorporated to describe the orientation of the B₀ field relative to the motion-corrected object; in other words the moving object appears to remain stationary relative to the gradient encoding field, as prospective motion correction has been applied. The result of the above is that the direction of the B₀ field appears to rotate and this knowledge is incorporated into the field calculation step of the method. This allows computation of the B₀ field at each point in the trajectory of the imaged object.

In a third step, corrections are applied to the spatially encoded MRI signals received, in order to obtain a high quality final image, using the knowledge of the specific field inhomogeneities at each time MRI signals were acquired. For example, corrections are applied to each image in an EPI time series corresponding to the time points where the field inhomogeneity was calculated, using the knowledge of field inhomogeneities at these time points. The corrections may include the correction of geometric image distortions [4]. Typically, to further improve image quality, the inventive corrections are applied to prospectively motion-corrected images.

It should be noted that the body part to be imaged may be relatively small, such as a particular brain region or a joint region, whereas the said part of the subject for which the susceptibility model is obtained (the potentially moving “object”) may be larger, e.g. including the complete head or limb parts connected to the joint, in order to track the relevant sources of field deviations effecting the imaging volume also in the vicinity of the imaging volume (note that the imaging volume, in general, approximately corresponds in size to the imaged body part). In some circumstances, it might be useful to take the complete subject (human or animal) as basis for the susceptibility model.

Further, in some circumstances, the imaged body part and the part of the subject for which the susceptibility model is obtained may be identical, e.g. when the complete head is imaged and only head motions are tracked and compensated for.

### Preferred Variants of the Inventive Method

In a preferred variant of the inventive method, step a) includes a segmentation of the said part of the subject, including the imaged body part, into a range of material types. Each part of the said part of the subject, including the imaged body part, is attributed to one material type, to which in turn a susceptibility value is attributed to. Typically, there are relatively few material types used in accordance with the invention, such as 6 or less.

Highly preferred is a further development of this variant wherein the segmentation applies only three different material types, e.g. “air”, “bone” and “water/other tissue”. This is particularly simple to compute, and can provide good corrections.

In an advantageous further development, the precision of segmentation in a region, in particular the number of material types and/or the spatial resolution, is adjusted based on an estimation of the influence of that region on the imaging volume. This is done using prior knowledge of tissue types, their susceptibilities and their distribution. Preferably, the segmentation becomes less detailed the farther the tissue is from the volume of interest (imaging volume), so computational capacity can be saved.

In a preferred variant, for optimizing the susceptibility model of step a), susceptibility-induced field deviations predicted with the susceptibility model are compared to those derived from an experimentally acquired B₀ field map, and the susceptibility model is altered to minimize the deviation between the two, in particular by using an iterative procedure. In other words, the susceptibility model is optimised by comparing a final predicted field (generated using the susceptibility model) to an acquired field. This process can be repeated iteratively until the susceptibility model produces a predicted field that is as close as possible to the acquired field. In this way, the accuracy of the inventive correction can be increased.

In another advantageous variant, for optimizing the susceptibility model of step a), residual artefacts in a final image are determined by way of a cost function, and the susceptibility model is altered to minimize the cost function, in particular in an iterative procedure. In other words, the susceptibility model is optimised by using a measure of artefacts in the final distortion-corrected images as a cost function. The cost function is iteratively minimised through modification of the susceptibility model. This also helps to increase the accuracy of the inventive correction.

An advantageous variant provides that in an additional step, field imperfections of the main magnetic field B₀ are quantified by experimentally mapping the main magnetic field B₀ without the subject. The extra (typically initial) step is performed to quantify imperfections in the main magnetic field, which are typically due to the magnet design limitations, imperfect shimming, or the presence of additional items not included in the model. This quantification is achieved either by forming a field map by imaging a large homogeneous phantom, or by using an external mapping technique such a Hall probe, a small MR frequency probe, or any other device capable of measuring the z-component of the main magnetic field. Many probes are used to acquire spatially-varying field information; alternatively, a single probe is used, but is moved to different locations to gain information about the field in these locations and produce the field map. This map is then added to later field predictions to account for the said imperfections. This variant can also improve the correction accuracy.

Another preferred variant provides that in an additional step main magnetic field imperfections are quantified by using an extra initial field map measurement and obtaining a reference field in the subject, comparing the result to a main magnetic field predicted using the susceptibility model and the known position and orientation of the imaged body part, and attributing the said field imperfections to the difference. Here main magnetic field imperfections are quantified by using an extra field map measurement to obtain the reference B₀ field in the object for a known position and orientation of the object. The predicted field in the object part is then subtracted from the measured field map to give a map of imperfections in the main magnetic field. As described in the above variant, this map of field imperfections is added to later field predictions.

In a preferred variant, echo planar imaging (EPI) is applied. Here prospective motion correction can be applied to update the imaging volume between slice or volume acquisitions and the inventive correction of susceptibility deviations, due to subject motion can be applied between recorded slices or volumes in a simple way.

In another preferred variant, an imaging technique that acquires k-space over multiple RF excitations is applied. In this case, correction is applied as part of the image reconstruction process, given knowledge of the B₀ inhomogeneities at the acquisition of every sample in k-space, rather than directly reconstructing using the fast Fourier transform. This can be achieved, for example, using the algorithm described in [5].

Preferred is a variant wherein shimming parameters are corrected between the acquisition of images to correct for time-varying susceptibility-induced field distortions, caused by motion of the subject. Further preferred is a variant where shimming parameters are corrected between RF excitations to correct for time-varying susceptibility-induced field distortions, caused by motion of the subject. Here the inventive method is combined with a “dynamic shimming” approach, as described e.g. in [6] (U.S. Pat. No. 6,509,735). The B₀ field estimation is performed during imaging and the shim settings that are available for dynamic switching are modified before the next spin excitation. This process dynamically reduces distortions in the B₀ field. The shim settings used are recorded and are later used together with the field estimation method to calculate the residual field inhomogeneities that remain uncorrected by the dynamic shim adjustment. After imaging, knowledge of these residual inhomogeneities is used for distortion correction or is used in advanced reconstruction methods, as described above. These variants reduce, in particular, the effect of the signal variations caused by susceptibility-induced T2* variations reported in [7].

An advantageous variant, which may in particular be combined with dynamic shimming, which can only provide low-order correction of field inhomogeneities, provides that residual artefacts are corrected in post processing. This can further improve the quality of the final images.

Further, a variant is preferred wherein tailored RF pulses, which generate a desired phase and amplitude modulation, are applied during imaging to provide correction for artefacts arising from predicted B₀ inhomogeneities. The use of such RF pulses for reduction of susceptibility effects in fMRI has been previously proposed by Glover and Lai [8]. Known field inhomogeneities are used to predict the resulting phase evolution in the imaged body part. The RF pulse is then designed so that it produces a phase response that is equal to the negative of the predicted phase evolution, thus compensating for the effect. This technique is described in more detail by Chen and Wyrwicz [9]. The combination of such RF pulses with the field estimation method proposed here allows for higher-order compensation of field inhomogeneities than can be achieved using the shim coils alone. The resulting images are then corrected for signal losses due to intravoxel dephasing. Distortions are then corrected for in the reconstruction, or by using the dynamic shimming approach described in the above.

In another advantageous variant, a change in shape of the said part of the subject, including the imaged body part, is taken into account in step b) in addition to changes in position and orientation. In this variant, the inventive method is extended to allow the correction of artefacts resulting from motion that is non-rigid and includes deformation of the said part of the subject, in particular of the imaged body part. The tracking system used is capable of quantifying deformations as well as rigid motion. This includes optical tracking tape, as described in [10], but also applies to any other suitable tracking system, including MR navigators. Tracking data are passed to a model, which mathematically represents the imaged object and the motion and deformations that occur during MR imaging. These deformations are applied to the above susceptibility model before estimation of the B₀ field inhomogeneities. This variant allows, in particular, for correction of susceptibility-related artefacts in abdominal and joint imaging, where motion is often non-rigid.

Finally, in a preferred variant, the position and orientation of the said part of the subject, including the imaged body part, is monitored by a separate tracking system, in particular including a single camera, a plurality of cameras, optical tracking tape, a tracking system using an RGR target or any structured marker, or by navigator echoes. Tracking systems separate from the MRI equipment require no extra imaging time to acquire tracking information. Tracking systems integrated into the MRI equipment (e.g. navigator echoes) may save space, reduce costs and be easier for the operator to use.

Further advantages can be extracted from the description and the enclosed drawing. The features mentioned above and below can be used in accordance with the invention either individually or collectively in any combination. The embodiments mentioned are not to be understood as exhaustive enumeration but rather have exemplary character for the description of the invention.

### DRAWING

The invention is shown in the drawing.

FIG. 1 illustrates an experimental setup used for prospective motion correction in magnetic resonance imaging, which is used with the present invention;

FIG. 2 illustrates the acquisition of tracking information from an imaged body part using a tracking system that is mounted inside the bore of the scanner, which is used with the present invention;

FIG. 3 is a flow chart summarising the steps used to generate the required susceptibility model of the object, in accordance with the invention;

FIG. 4 is a flow chart summarising the steps in the inventive method, given the susceptibility model formed as described in FIG. 3.

In the following, the inventive method is described by way of example, wherein the imaging is based on echo planar imaging (EPI) of the human head as an example of an imaged body part. It is noted that the invention is neither restricted to the particular measures or pulse sequences used in the example, nor to imaging of the human brain. In addition, EPI distortion correction is used as an example of a correction method that can be used with the invention. Again, it is noted that the invention is not restricted to use with solely EPI distortion correction.

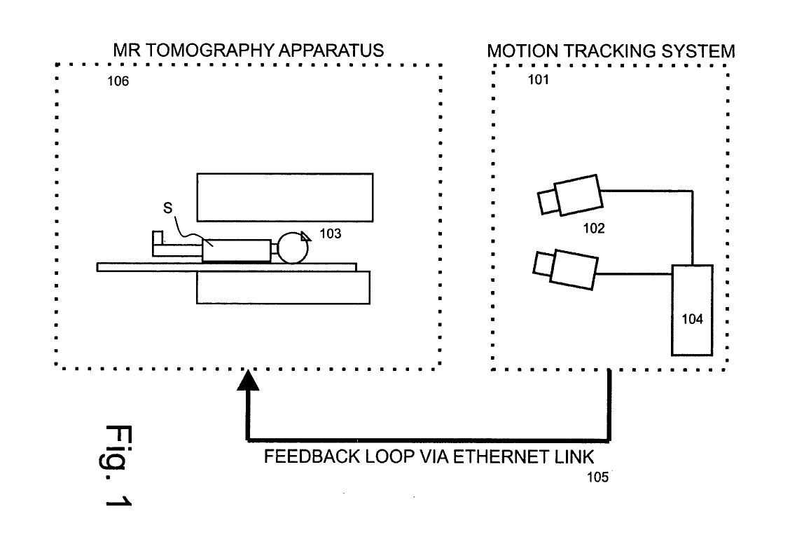

FIG. 1 shows a possible arrangement of the hardware required to perform prospective motion correction; the information obtained with that arrangement is also used to perform the inventive method, in particular step b) of the inventive method.

The position and orientation (in six degrees of freedom) of the head of the subject S is determined using an optical motion tracking system 101 using one or more cameras 102 (two cameras are used in this example). In the example shown, the cameras track a target consisting of reflective spheres 103 attached to the head; however, any suitable target could be used, including a retro-grate reflector system as described in US patent 2007/0280508. The motion tracking information is processed by a computer 104 and the tracking information is then passed via an Ethernet link 105 to the magnetic resonance apparatus 106. In this example, the tracking data must be converted from tracking system coordinates into the same coordinates used by the MRI apparatus. This is performed using the method in [2]. Finally, the slice position and slice orientation are adjusted to follow the motion of the head, as described in [2].

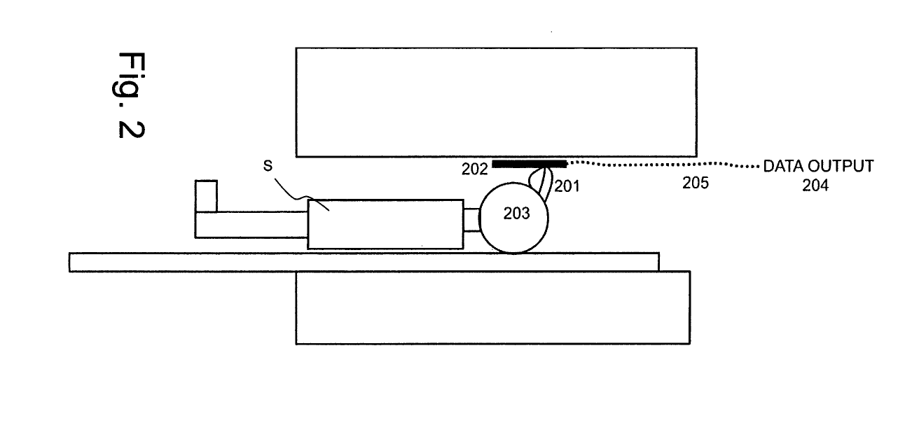

FIG. 2 shows a further arrangement that is used to acquire data concerning the position, orientation, and, optionally, the shape and/or changes in shape of the imaged body part, to be used with the invention. In this case, the tracking system is placed inside the bore of the MRI scanner. This has the advantage of not requiring optical line-of-sight from outside the scanner bore to the imaged body part (here the head of the subject S). As an example in FIG. 2, optical tracking tapes 201, as reported in [10], are attached to a fixed reference point 202 inside the scanner and then to the body part being imaged 203.

In the case of rigid-body tracking, where position and orientation information in six degrees of freedom is required, the individual tapes are connected to a rigid attachment mounted on the head; in the case of non-rigid tracking, where deformation information about the imaged body part is required, the individual tapes are attached separately to the body or wrapped around the imaged body part. In this manner, deformation information can be obtained, which is then used in the deformation model of the body part in question.

The in-bore tracking system used need not be optical tracking tape; rather, any tracking system that can be placed in the bore of the magnet can be used. This includes MR-compatible cameras, combined with the RGR tracking target in [11] (U.S. Pat. No. 5,936,722), or using a conventional marker, such as that reported in [12]. Alternatively, stereo vision, or a time-of-flight depth-sensing camera, may be used for tracking without requiring any marker at all. Furthermore, the tracking system used need not be optical: MR-based systems, including so-called ‘active markers’ [13] can be used to acquire the position and rotation (orientation) of the object. Note that when the tracking system is an MRI-based system, a coordinate transform as discussed in the description of FIG. 1 is not required.

When using a tracking system located inside the bore of the magnet, such as that shown in FIG. 2, the data 204 from the system must be transmitted from within the bore of the magnet to the room outside the bore. It is important that the data transmission system and the MR system do not interfere with each other. In the example shown here, this is achieved using an MR-compatible cable 205, consisting of optical fibre.

Note that in both illustrated examples of FIG. 1 and FIG. 2, the imaged body part and the part of the subject whose position and orientation is monitored and the susceptibility model is applied for (“object”), here the head, are identical.

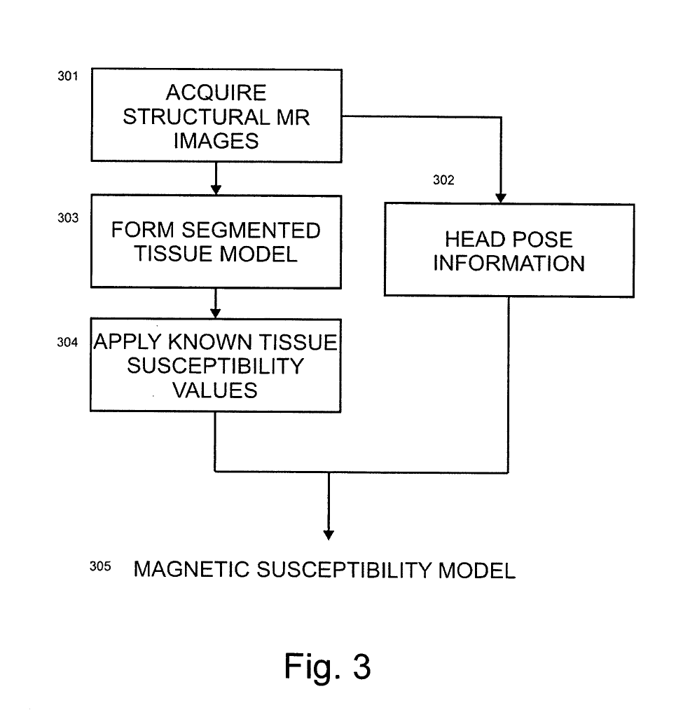

FIG. 3 illustrates the method used to construct a susceptibility model for the object by way of example. First, the object is imaged 301 using a structural imaging sequence. Head position and orientation information 302 from the tracking system is recorded during this process. The image data are then segmented 303 into various tissue types (or, more generally, material types).

Prior knowledge of likely values from a brain atlas (a map of the brain showing the probable tissue type at any given location) may be used to make this procedure more robust. Known magnetic tissue susceptibility values (from [14], for example) are then applied for each tissue type 304. The final result is a magnetic susceptibility model 305 matching the subject (or the relevant part of the subject) and in known coordinates in the frame of reference of the MRI apparatus.

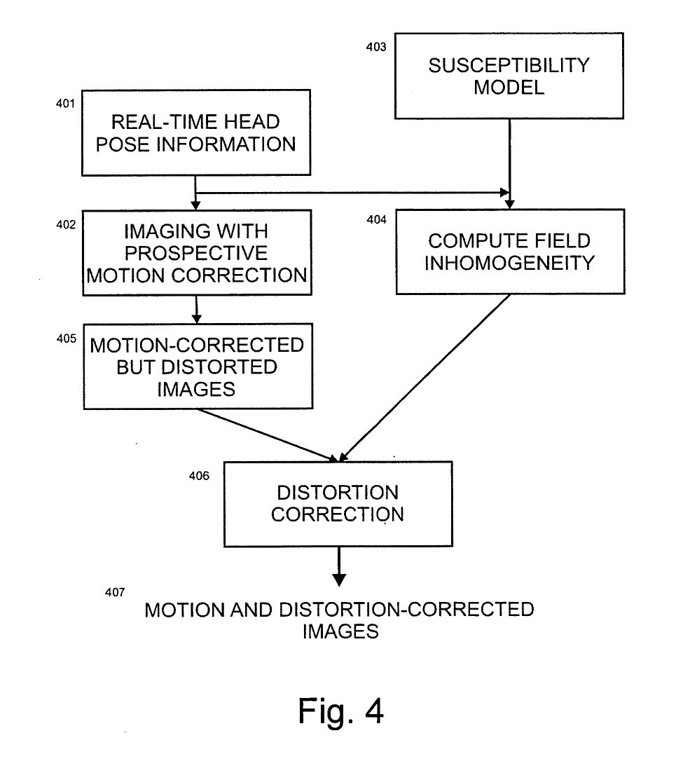

FIG. 4 shows the steps in the inventive method, given the susceptibility model formed as described in FIG. 3. MR imaging is performed while simultaneous using the motion tracking system 401 to provide head position and orientation data for prospective motion correction 402, such that the imaging volume is updated before each spin excitation. Head position and orientation information is recorded for each slice. After imaging, the recorded head position and orientation information is combined with the susceptibility model 403 to calculate the B₀ field for each slice 404. This step is based on the expression stated in [15],

$$Δ {B}_{0} ( k ) = {B}_{0} [ \frac{1}{3} - \frac{{k}_{z}^{2}}{{k}_{x}^{2} + {k}_{y}^{2} + {k}_{z}^{2}} ] \cdot χ ( k ) , \qquad ( 1 )$$

which gives the field inhomogeneity ΔB₀(k) in k-space, given the nominal field strength, B₀, and the susceptibility distribution in k-space, X(k). The field distribution in image-space is recovered by inverse Fourier transformation of Eq. (1). Alternative field calculation methods, such as the perturbation method described in [16], can be used instead of that mentioned above.

Combining prospective motion correction with the above field estimation method requires the incorporation of the apparent change in the B₀ field orientation. This can be done by applying the corresponding coordinate transform to the k-space coordinates in Eq. (1). As an example, for an apparent rotation of the B₀ field around the x-axis of α°, the Fourier transform of the induced field is then,

$$Δ {\tilde{B}}_{0}^{z} ( k , α ) = {B}_{0} [ \frac{1}{3} - \frac{{( {k}_{z} cos α + {k}_{y} sin α )}^{2}}{{k}_{x}^{2} + {k}_{y}^{2} + {k}_{z}^{2}} ] \cdot \tilde{χ} ( k ) \qquad ( 2 )$$

Again, inverse Fourier transformation yields the field inhomogeneities in image-space in rotated coordinates. Alternatively, the susceptibility model may be rotated and shifted, and the calculation is performed in original coordinates.

Accurate numerical evaluation of the above equations requires a sufficiently large computational volume of the input susceptibility model or “fold back” artefacts occur because of the natural periodicity of the discrete Fourier transformation. Thus, the computational volume is first zero-padded to ensure that the induced fields have decayed to zero at the boundaries of the volume and that no artefacts occur.

The B₀ field for each slice 404, computed as described above, together with the motion-corrected images 405 is used by a distortion correction algorithm to obtain the fully-corrected images 407.

### LITERATURE

[1] S. Thesen, O. Heid, E. Mueller, and L. R. Schad, “Prospective acquisition correction for head motion with image-based tracking for real-time fMRI,” Magn Reson Med, vol. 44, pp. 457-463, September 2000.
[2] M. Zaitsev, C. Dold, G. Sakas, J. Hennig, and O. Speck, “Magnetic resonance imaging of freely moving objects: prospective real-time motion correction using an external optical motion tracking system,” Neuroimage, vol. 31, pp. 1038-1050, July 2006.
[3] K. M. Koch, X. Papademetris, D. L. Rothman, and R. A. de Graaf, “Rapid calculations of susceptibility-induced magnetostatic field perturbations for in vivo magnetic resonance,” Phys Med Biol, vol. 51, pp. 6381-402, Dec 21, 2006.
[4] P. Jezzard and R. S. Balaban, “Correction for geometric distortion in echo planar images from BO field variations,” Magn Reson Med, vol. 34, pp. 65-73, July 1995.
[5] K. P. Pruessmann, M. Weiger, P. Bornert, and P. Boesiger, “Advances in sensitivity encoding with arbitrary k-space trajectories,” Magn Reson Med, vol. 46, pp. 638-51, October 2001.
[6] E. Mueller and S. Thesen, 2003, “Method for operating a magnetic resonance tomography apparatus with shim coil adjustment dependent on positional changes of the imaged region,” U.S. Pat. No. 6,509,735.
[7] E. C. Caparelli, D. Tomasi, and T. Ernst, “The effect of small rotations on R2* measured with echo planar imaging,” NeuroImage, vol. 24, pp. 1164-1169, 2005.
[8] G. H. Glover and S. Lai, “Reduction of susceptibility effects in fMRI using tailored RF pulses,” in Proceedings of the International Society for Magnetic Resonance in Medicine, Sydney, Australia, 1998, p. 298.
[9] N. Chen and A. M. Wyrwicz, “Removal of intravoxel dephasing artifact in gradient-echo images using a field-map based RF refocusing technique,” Magn Reson Med, vol. 42, pp. 807-12, October 1999.
[10] J. Maclaren, R. Boegle, J. Hennig, and M. Zaitsev, “Prospective motion correction in MRI using optical tracking tape,” in 26th Annual Scientific Meeting of the ESMRMB, 2009.
[11] B. S. R. Armstrong and K. B. Schmidt, 1999, “Apparatus and method for determining the angular orientation of an object,” U.S. Pat. No. 5,936,722.
[12] M. Aksoy, R. Newbould, M. Straka, S. Holdsworth, S. Skare, J. Santos, and R. Bammer, “A Real Time Optical Motion Correction System Using a Single Camera and 2D Marker,” in Proceedings 16th Scientific Meeting, International Society for Magnetic Resonance in Medicine, 2008, p. 3120.
[13] M. B. Ooi, S. Krueger, W. J. Thomas, S. V. Swaminathan, and T. R. Brown, “Prospective real-time correction for arbitrary head motion using active markers,” Magn Reson Med, vol. 62, pp. 943-54, October 2009.
[14] J. F. Schenck, “The role of magnetic susceptibility in magnetic resonance imaging: MRI magnetic compatibility of the first and second kinds,” Med Phys, vol. 23, pp. 815-50, June 1996.
[15] R. Boegle, J. Maclaren, and M. Zaitsev, “Prediction of susceptibility-induced artefacts for prospective motion correction,” in Proceedings 17th Scientific Meeting, International Society for Magnetic Resonance in Medicine, Honolulu, 2009, p. 3075.
[16] M. Jenkinson, J. L. Wilson, and P. Jezzard, “Perturbation method for magnetic field calculations of nonconductive objects,” Magn Reson Med, vol. 52, pp. 471-7, September 2004.

1-16. (canceled)

17. A method of magnetic resonance imaging (MRI), wherein a body part of an animal or human subject in an imaging volume is imaged by acquiring a plurality of spatially-encoded MR signals from the imaging volume where susceptibility-induced field deviations reduce a homogeneity of a main magnetic field B₀, and wherein a prospective motion correction is applied, updating the imaging volume between the acquisition of the spatially-encoded MR signals based on a monitored position of the body part, the method comprising the steps of:
a) forming a susceptibility model of at least part of the subject, including an imaged body part, using a structural magnetic resonance image of that part of the subject and/or prior knowledge of an anatomy of the subject;
b) computing susceptibility-induced field deviations present in the imaging volume at each time MR signals are acquired using the susceptibility model and the knowledge of a monitored position and monitored orientation of the part of the subject at those times at which MR signals are acquired; and
c) using information about the susceptibility-induced field deviations derived in b) for image correction or for correction of image distortions and/or intensity modulations.

18. The method of claim 17, wherein step a) includes segmentation of the part of the subject, including the imaged body part, into a range of material types.

19. The method of claim 18, wherein the segmentation applies to only three different material types or to air, bone and water/other tissue.

20. The method of claim 18, wherein a precision of segmentation in a region, a number of material types and/or a spatial resolution, is adjusted based on an estimation of influence of that region on the imaging volume.

21. The method of claim 17, wherein, for optimizing the susceptibility model of step a), susceptibility-induced field deviations predicted with the susceptibility model are compared to those derived from an experimentally acquired B₀ field map and the susceptibility model is altered to minimize a deviation between predicted and acquired B₀ field deviations.

22. The method of claim 21, wherein an iterative procedure is used.

23. The method of claim 17, wherein, for optimizing the susceptibility model of step a), residual artefacts in a final image are determined by way of a cost function and the susceptibility model is altered to minimize the cost function or to minimize the cost function in an iterative procedure.

24. The method of claim 17, wherein, in an additional step, field imperfections of the main magnetic field B₀ are quantified by experimentally mapping the main magnetic field B₀ without the subject.

25. The method of claim 17, wherein, in an additional step, main magnetic field imperfections are quantified using an extra initial field map measurement and obtaining a reference field in the subject, comparing a result to a main magnetic field predicted using the susceptibility model and a known position and orientation of the imaged body part and attributing field imperfections to a difference between a predicted a measured main magnetic field.

26. The method of claim 17, wherein echo planar imaging (EPI) is applied.

27. The method of claim 17, wherein an imaging technique is applied which acquires k-space over multiple RF excitations.

28. The method of claim 17, wherein shimming parameters are corrected between acquisition of images to correct for time-varying susceptibility-induced field distortions caused by motion of the subject.

29. The method of claim 17, wherein shimming parameters are corrected between RF excitations to correct for time-varying susceptibility-induced field distortions caused by motion of the subject.

30. The method of claim 17, wherein residual artefacts are corrected in post processing.

31. The method of claim 11, wherein residual artefacts are corrected in post processing.

32. The method of claim 12, wherein residual artefacts are corrected in post processing.

33. The method of claim 17, wherein tailored RF pulses, which generate a desired phase and amplitude modulation, are applied during imaging to provide correction for artefacts arising from predicted B₀ inhomogeneities.

34. The method of claim 17, wherein a change in shape of the part of the subject, including the imaged body part, is taken into account in step b) in addition to changes in position and orientation.

35. The method of claim 17, wherein a position and orientation of the part of the subject, including the imaged body part, is monitored by a separate tracking system, a single camera, a plurality of cameras an optical tracking tape, a tracking system using an RGR target or any structured marker or by navigator echoes.
